(19) United States
(12) Patent Application Publication
(10) Pub. No.: US 2009/0205986 A1
(43) Pub. Date: Aug. 20, 2009

(54) MEDICAL DEVICE PACKAGING TRAY

(75) Inventors: Brett O. Baker, Ellettsville, IN (US); Brian K. Long, Blommington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/328,556

(22) Filed: Dec. 4, 2008

Related U.S. Application Data
(60) Provisional application No. 60/992,964, filed on Dec. 6, 2007.

(51) Int. Cl. A61B 19/02

(52) U.S. Cl. 206/363

(57) ABSTRACT

A packaging tray for a medical device having a substantially flat configuration with at least one surface thereof comprising a coating of an agent disposed thereupon and an uncoated portion extending laterally therefrom. The packaging tray comprises a container unit having top and bottom portions that define a protective chamber therebetween configured to receive the coated portion of the medical device. The container unit is further configured to include an anchoring region disposed lateral to the protective chamber and between the top and bottom portions. When the medical device is disposed within the container unit, the uncoated portion is at least partially received and secured within the anchoring region, and the coated portion is maintained within the protective chamber, such that the coating of the agent is prevented from contacting the top and bottom portions of the container unit.

### CROSS-REFERENCE TO RELATED APPLICATIONS

This application claims the benefit of U.S. Provisional Application Ser. No. 60/992,964, filed Dec. 6, 2007, entitled “Medical Device Packaging Tray”, the entire contents of which are incorporated by reference.

### TECHNICAL FIELD

The invention relates to a packaging tray for a medical device, and in particular to a packaging tray for a surgical staple line reinforcement material having an adhesive or other coating agent disposed thereon.

### BACKGROUND OF THE INVENTION

Surgical stapler devices are designed to simultaneously cut and seal an extended segment of tissue in a patient. Typically, a surgical stapler comprises two stapler arms, one arm containing two or more lines of staples and a second arm containing an anvil structure to bend each of the staples into a closed position. For many applications, a surgical blade is included in the device to quickly sever tissue between the lines of staples. Those stapler devices employing a cutting blade are referred to as “anastomotic staplers” and those used without a cutting blade are referred to as “non-anastomotic staplers.”

For some procedures, the use of bare staples, with the staples in direct contact with the patient's tissue, is generally acceptable. The integrity of the tissue itself will normally prevent the staples from tearing out of the tissue and compromising the seam before healing has occurred. In certain circumstances, however, the tissue that is being sealed is too fragile to securely hold the staples in place. In these instances, the tissue will tend to rip at or near the staple lines, which may slow healing and possibly leading to serious complications.

One area where fragile tissue is of particular concern is the use of stapler devices in lung tissue, and especially lung tissue that is affected by emphysema or similar diseased conditions. Diseased lung tissue is very fragile and, in extreme cases, will readily tear through unprotected staple lines. With the growing use of surgical staplers in operations on diseased lung tissue such as bullectomies and volume reduction procedures, it has become increasingly important to develop some reliable means to protect fragile tissue from tissue tears due to surgical staples or surgical stapling procedures. Moreover, when staples are used, it is desirable to reduce any leakage around the staples.

One known protective measure involves the use of reinforcement or bolster material, wherein the staples are driven through both the reinforcement material and the patient's tissue. The reinforcement material inhibits tearing of the tissue or leakage around the staples. The use of staple line reinforcement is typically accomplished by temporarily affixing the reinforcement material to each arm of the stapler prior to stapling the patient's tissue. The staples are then driven through the reinforcement material and the patient's tissue, sandwiching the tissue between two layers of reinforcement material. An adhesive or other coating agent is often applied to the reinforcement material to help bond the reinforcement material to the tissue or perform some other task. Examples of staple line reinforcement materials are disclosed in US Publication No. 2004/0093029, entitled “Apparatus and Method for Producing a reinforced Surgical Staple Line”, and US Publication No. 2007/0027472, entitled “Medical Devices and Methods Useful for Applying Bolster Material”, the entire contents of which are hereby incorporated by reference.

An adhesive or other coating agent is often utilized to temporarily affix the reinforcement or bolster material to the arms of the stapler. The coating agent is typically supplied to the user separately from the reinforcement material as part of a kit. The user must then apply the coating agent to the reinforcement material just prior to affixing the material to the stapler arms. The task can be time consuming, and often results in contamination of the reinforcement material. In addition, the user may be unable to apply the coating agent to the reinforcement material uniformly or in the appropriate amount. Thus, it is desirable to pre-coat the reinforcement material at the time of manufacture, and prior to delivery to the end user. However, the pre-coated reinforcement material must be protected from inadvertent contact with packaging or other materials during transport, storage and delivery to the user. Thus, it is desirable to provide a packaging tray for a medical device, such as a pre-coated staple line reinforcement material, that protects the device from contamination and inadvertent contact with the packaging material prior to use. These and other purposes of the present invention will become evident from review of the following specification.

### BRIEF SUMMARY OF THE INVENTION

Accordingly, the present invention provides a packaging tray for a medical device having features that resolve or improve upon one or more of the above-described drawbacks.

In a first aspect of the present invention, the packaging tray is adapted to hold a medical device comprising a substantially flat configuration and having a portion of at least one surface thereof comprising a coating of an agent disposed thereupon. The medical device further comprises an uncoated portion thereof extending laterally from the portion of the at least one surface comprising the agent. In one particular embodiment, the medical device comprises an implantable material removably disposed on an applicator strip. The applicator strip comprises the laterally extending uncoated portion, and the coating of the agent is disposed on the implantable material.

The packaging tray comprises a container unit having a top portion and a bottom portion that defines a protective chamber therebetween configured to receive the coated portion of the medical device. The container unit is further configured to include an anchoring region disposed lateral to the protective chamber and between the top and bottom portions. When the medical device is disposed within the container unit, the uncoated portion is at least partially received and secured within the anchoring region, and the coated portion is maintained within the protective chamber, such that the at least one surface comprising the coating of the agent is prevented from contacting the top and bottom portions of the container unit. In one particular embodiment, the top and bottom portions of the container unit comprise separate and identical tray portions that are connected together by a plurality of attachment portions. The tray portions may also comprise a translucent material, such as transparent plastic, to permit viewing of a medical device disposed therein.

These and other advantages, as well as the invention itself, will become apparent in the details of construction and operation as more fully described below. Moreover, it should be appreciated that several aspects of the invention can be used with packaging for other types of staple line reinforcement materials or medical devices.

### BRIEF DESCRIPTION OF SEVERAL VIEWS OF THE DRAWINGS

Embodiments of the present invention will now be described by way of example with reference to the accompanying drawings, in which:

FIG. 1 illustrates a perspective view of an exemplary embodiment of the packaging tray of the present invention;

FIG. 2 is a top view of a medical device configured for use with the packaging tray of the present invention;

FIG. 3 is a top view of the packaging tray illustrated in FIG. 1;

FIG. 4 is a cross-sectional view of the packaging tray taken along line 4-4 of FIG. 3;

FIG. 5 is a cross-sectional view of the packaging tray taken along line 5-5 of FIG. 3;

FIG. 6 is a cross-sectional view of the packaging tray taken along line 6-6 of FIG. 3;

FIG. 7 is a cross-sectional view of the packaging tray taken along line 7-7 of FIG. 3; and

FIG. 8 is an exploded side view of the top and bottom portions of the packaging tray and medical device.

### DETAILED DESCRIPTION

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention, such as conventional details of fabrication and assembly.

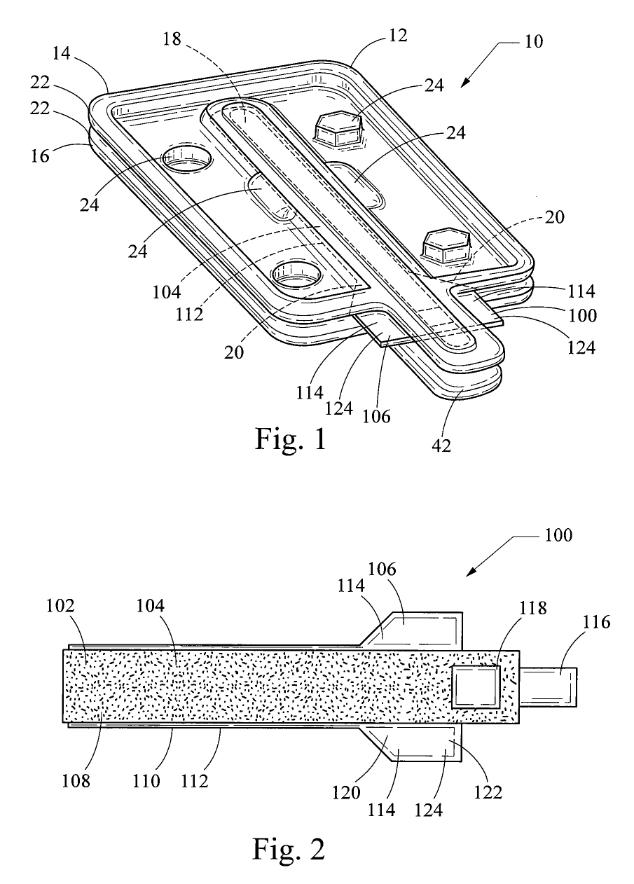

In general, FIG. 1 illustrates an exemplary embodiment of the packaging tray 10 of the present invention. The packaging tray 10 is adapted to hold a medical device 100 therein. In particular, the packaging tray 10 is adapted to hold and secure a medical device 100 in a partially suspended manner such that certain portions of the medical device 100 are protected from contamination and prevented from coming into contact with the packaging tray 10. In addition, the packaging tray 10 is configured to allow the medical device 100 to be easily removed from the packaging tray 10.

One embodiment of the medical device 100 for use with the packaging tray 10 is illustrated in FIG. 2. In general, the medical device 100 comprises a substantially flat configuration. One portion of the medical device 100 includes at least one surface 102 having a coating 104 of an agent disposed thereupon. The medical device 100 further comprises an uncoated portion 106 thereof extending laterally from the coated portion 104 (i.e., the at least one surface 102 comprising the agent). As will be explained in greater detail below, the packaging tray 10 is configured to hold the medical device 100 in a partially suspended manner such that the coated portions 104 is protected from contamination and prevented from coming into contact with the packaging tray 10.

In the particular embodiment illustrated, the medical device 100 comprises an implantable material 108 removably disposed on an applicator strip 110. The applicator strip 110 comprises a central portion 112, two laterally extending portions or wings 114, and a removable tab 116 extending from the rearward end thereof. The central portion 112 is sized and configured to support the implantable material 108 thereon, and is preferably sized and configured to align with the arms of a surgical stapler (not shown) or other device on to which the implantable material 108 is to be removably attached. In particular, the central portion 112 preferably has a width that is equal to that of the implantable material 108 and the arms of the surgical stapler.

The removable tab 116 is configured to engage slots 118 in the ends of the implantable material 108 so as to secure it to the applicator strip 110. In particular, the implantable material 108, which comprises a single strip of bolster material, is secured to the applicator strip 110 by first folding the bolster material over the central portion and then passing the slot 118 on each end of the bolster material over the tab 116. The implantable material 108 may subsequently be removed or separated from the applicator strip 110 by tearing or otherwise removing the tab 116 from the applicator strip 110. This typically occurs at the time the implantable material 108 is affixed to the arms of the surgical stapler.

The laterally extending wings 114 provide a grasping area for the user. In particular, the user may grasp the wings 114 and manipulate the position of the applicator strip 110 without engaging the or otherwise coming into contact with the implantable material 108. As will be explained in greater detail below, the laterally extending wings 114, which generally comprise the uncoated portion of the medical device 100, also provide a mechanism for securing the applicator strip 110 to the packaging tray 10. Although two laterally extending wings 114 are shown, fewer or more than two wings can be utilized. In addition, it should be appreciated that the shape of the laterally extending wings 114 is not critical to the present invention.

In the embodiment illustrated, the applicator strip 110 comprises a compressible material such as polymer foam (e.g., styrene or Styrofoam). A compressible material facilitates attachment of the implantable material 108 to the arms of the surgical stapler by allowing uniform pressure to be applied by the arms to the implantable material 108 during attachment thereof.

The implantable material 108 comprises a reinforcement or bolster material, such as a layer of dried, collagenous extracellular matrix (ECM) material, suitable for use with surgical staplers. The coating 104 of the agent is disposed on the exterior surface of the implantable material 108 (i.e., the surface that is opposite the surface that faces the central portion 112 of the applicator strip 110). One example of a preferred coating 104 is a dried, reversible gelatin based adhesive coating that is adapted to allow the implantable material 108 to be temporarily affixed to the arms of the surgical stapler. Other types of coatings 104 may include medicaments, pharmacologically active materials and/or biological materials. In addition, coatings 104 may also be applied to the interior surface of the implantable material 108 (i.e., the surface which faces or engages the central portion 112 of the applicator strip 110). As explained in background section above, it is preferable to apply the coating 104 to the implantable material 108 at the time the medical device 100 is manufactured. However, the coating 104 must be protected from contamination and prevented from coming into contact with packaging or other surfaces during transport, storage and delivery to the user. As will be explained in greater detail below, the packaging tray 10 of the present invention is configured to protect the coating 104, as well as the implantable material 108 and the applicator strip 110, subsequent to the initial manufacture of the medical device 100.

Additional details regarding the applicator strip 110, implantable material 108 and coating 104 are disclosed in US Publication No. 2007/0027472, entitled “Medical Devices and Methods Useful for Applying Bolster Material”, the entire contents of which are hereby incorporated by reference. Alternative configurations for these components are also disclosed in the above-referenced publication, as well as in US Publication No. 2004/0093029, entitled “Apparatus and Method for Producing a reinforced Surgical Staple Line”. It should be understood by those skilled in the art that the packaging tray 10 of the present invention may be modified or re-configured to accommodate one or more of these alternative configurations without departing from the scope of the invention. In addition, it should be understood by those skilled in the art that the packaging tray 10 of the present invention may be modified, re-configured or otherwise adapted to accommodate other types of medical devices 100 having coated portions that require protection from inadvertent contact with the packaging or other surfaces, and that the packing tray 10 of the present invention is not limited to medical devices 100 for use with surgical staplers.

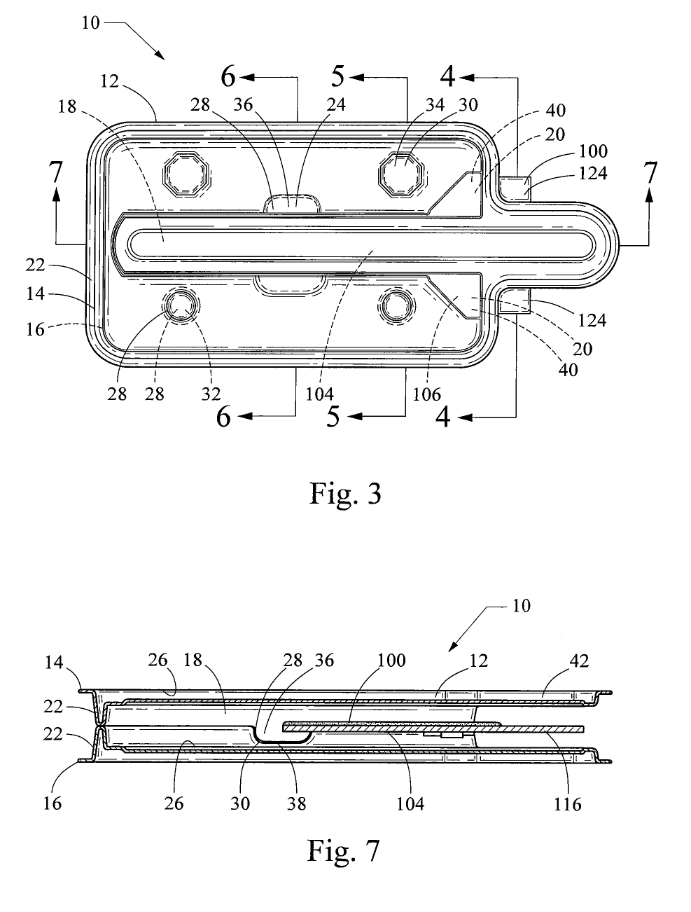

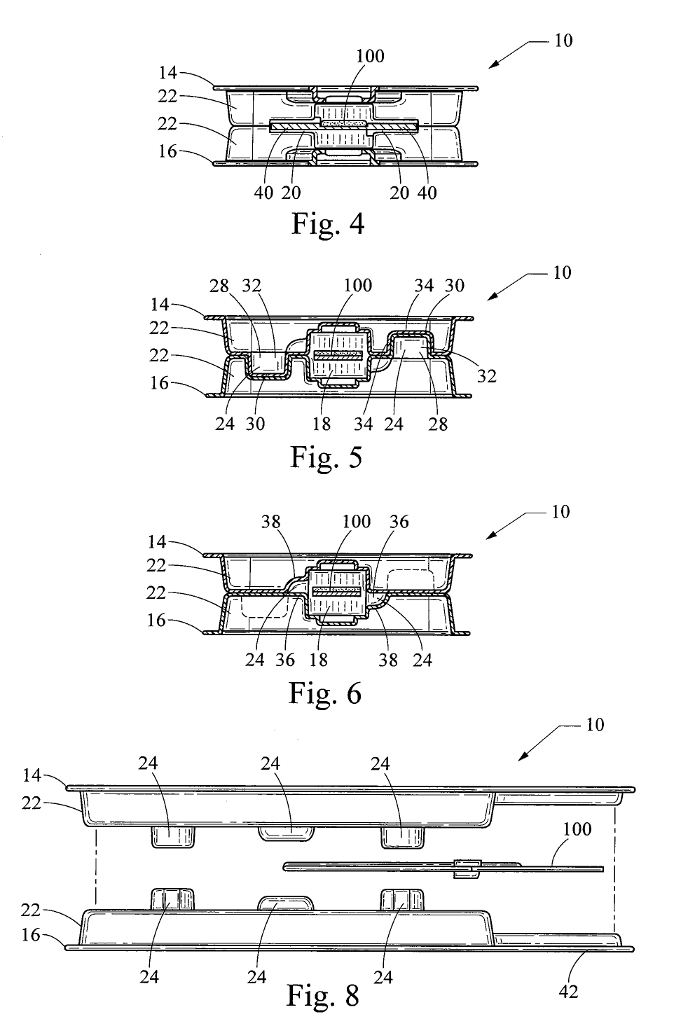

An embodiment of the packaging tray 10 of the present invention is illustrated in FIGS. 1 and 3-8, wherein FIG. 1 is a perspective view of the packaging tray 10, FIG. 3 is a top view of the packaging tray 10, FIGS. 4-7 are cross-sectional views of the packaging tray 10 taken along respective lines of FIG. 3, and FIG. 8 is an exploded side view of the packaging tray 10. The packaging tray 10 comprises a container unit 12 having a top portion 14 and a bottom portion 16 that define a protective chamber 18 therebetween. As will be explained in greater detail below, the protective chamber 18 is configured to receive the coated portion 104 of the medical device 100. The container unit 12 is further configured to include an anchoring region 20 disposed lateral to the protective chamber 18 and between the top and bottom portions 14, 16. When the medical device 100 is disposed within the container unit 12, the uncoated portion 106 is at least partially received and secured within the anchoring region 20, and the coated portion 104 is maintained within the protective chamber 18, such that the at least one surface comprising the coating 104 of the agent is prevented from contacting the top and bottom portions 14, 16 of the container unit 12.

In the particular embodiment illustrated, the top and bottom portions 14, 16 of the container unit 12 comprise separate and identical tray portions 22 that are connected together by a plurality of attachment portions 24. In particular, and as best seen in FIG. 8, the tray portions 22 are configured so that one tray portion 22 (e.g., the top portion 14) may be turned upside down and engaged with the other tray portion 22 (e.g., the bottom portion 16). Thus, the protective chamber 18 and the anchoring region 20 are each formed by various depressions in the interior surface 26 of each tray portion 22. Likewise, the attachment portions 24 are formed by a combination of projections and depressions disposed on the interior surface 26 of each tray portion 22. In particular, the attachment portions 24 comprise a plurality of male attachment members 28 that are configured to lockingly engage a plurality of female attachment sockets 30.

In the particular embodiment illustrated, each tray portion 22 comprises a pair of circular shaped male attachment members 32 along one side thereof, a pair of octagon shaped female attachment sockets 34 along the opposite edge thereof, a single elongate male attachment member 36 adjacent to one edge of the protective chamber 18, and a single elongate female attachment socket 38 along the opposite edge of the protective chamber 18. Thus, the pair of circular shaped male attachment members 32 of one tray portion 22 is configured to engage the pair of octagon shaped female attachment sockets 34 of the other tray portion 22. Likewise, the single elongate male attachment member 36 of one tray portion 22 is configured to engage the single elongate female attachment socket 38 of the other tray portion 22. The arrangement provides a total of six attachment portions 24 for connecting the two tray portions 22 together.

Of course, it should be understood that fewer or additional attachment portions 24, or differently shaped attachments portions 24, may be utilized to connect the tray portions 22 together. Likewise, the attachment portions 24 may comprise a non-mechanical mechanism such as an adhesive for connecting the tray portions 22 together. Furthermore, the top and bottom portions 14, 16 of the container unit 12 may be formed as a unitary structure as opposed to a plurality of components that have been assemble together. Finally, it should be understood that the top and bottom portions 14, 16 do not need to be formed from identical tray portions 22, but may alternatively comprise different shaped tray portions 22. For example, all of the male attachment members 28 may be formed on one tray portion 22 (e.g., the top portion 14), while all of the female attachment sockets 30 may be formed on the other tray portion 22 (e.g., the bottom portion 16).

As best seen in FIGS. 3, 5 and 6, the protective chamber 18 has a depth, as measured between the interior surface of the top and bottom portions 14, 16, that is substantially greater than the thickness of the coated portion 104 of the medical device 100. In particular, the depth of the protective chamber 18 is substantially greater than the combined thickness of the central portion 112 of the applicator strip 110 and the implantable material 108. This arrangement prevents the coated portion 104 of the medical device 100 (i.e., the adhesive coating on the exterior surface of the implantable material 108) from coming into contact with the interior surface of the top and bottom portions 14, 16 of the packaging tray 10. In the particular embodiment illustrated, the protective chamber 18 comprises a width that is greater than the width of the coated portion 104 of the medical device 100 (i.e., the width of the central portion 112 of the applicator strip 110 and/or the width of the implantable material 108). This arrangement likewise prevents the coated portion 104 adhesive coating on the exterior surface of the implantable material 108 of the medical device 100 from coming into contact with the interior surface of the top and bottom portions 14, 16 of the packaging tray 10. Alternatively, the width of the protective chamber 18 may be approximately equal to the width of the coated portion 104 of the medical device 100 so as to maintain alignment of the medical device 100 within the protective chamber 18 by, for example, engaging the outside edges of the central portion 112 of the applicator strip 110. The width, depth and length of the protective chamber 18 is preferably sized and configured so as to accommodate medical devices 100 of various sizes and shapes.

As best seen in FIGS. 3 and 4, the anchoring region 20 is configured to receive, engage and secure the uncoated portion 106 of the medical device 100 therein. In particular, the anchoring region 20 is configured to engage at least a portion of the laterally extending wings 114 of the applicator strip 110 in such a manner so as to prevent the medical device 100 from inadvertently falling out of or being unintentionally removed from the packaging tray 10. In the preferred embodiment, the anchoring region 20 has a depth, as measured between the interior surface of the top and bottom portions 14, 16, that is slightly less than the thickness of the uncoated portion 106 of the medical device 100 (i.e., the thickness of the wings 114 of the applicator strip 110) so as to cause frictional engagement therebetween. This arrangement allows the coated portion 104 of the medical device 100 (i.e., the central portion 112 of the applicator strip 110 and the implantable material 108 disposed thereon) to be suspended within the protective chamber, thereby preventing the coated portion 104 from coming into contact with the interior surfaces of the top and bottom portions 14, 16 of the container unit 12.

As best seen in FIGS. 1 and 3, the anchoring region 20 is preferably configured to engage only a portion of the uncoated portion 106 of the medical device 100. For example, the anchoring region 20 comprises a pair of anchoring receptacles 40 disposed adjacent to each side of the protective chamber 18, wherein each anchoring receptacle 40 has a planar width and/or length that is less than that of each wing 114 of the applicator strip 110 so that a first portion 120 of each wing 114 is disposed within an anchoring receptacle while a second portion 122 of each wing 114 is exposed to the exterior of the packaging tray 10. The second portion 122 of each wing 114 is exposed to the exterior so as to provide a grasping area 124 that may be grasped by the user when removing the medical device 100 from the packaging tray 10. Although the anchoring region 20 is configured to frictionally engage the uncoated portion 106 of the medical device 100, it should be understood that other types of engagement may be employed to temporarily secure the medical device 100 to the packaging tray 10. Likewise, the anchoring region 20 may comprise other sizes, shapes and configurations depending of the size, shape and configuration of the medical device 100 to be secured, or the manner of securement desired.

As best seen in FIGS. 1 and 3, the container unit 12 further comprises a protective tab 42 that is configured to protect a portion of the medical device 100 from damage or inadvertent contact. In particular, the protective tab 42 is configured to protect the portion of the medical device 100 extending outwardly from the protective chamber 18. For example, the protective tab 42 is configured to protect the tab 116 of the applicator strip 110, as well as the ends of the implantable material 108 containing the slots 118. In other words, the protective tab 42 defines an interior area that is an extension of the protective chamber 18. The protective tab 42, and the protective area therebetween, is formed by the top and bottom portions 14, 16 of the tray portions 22.

In the embodiment illustrated, the tray portions 22 comprise a translucent material, such as transparent plastic, to permit viewing of a medical device 100 disposed therein. In particular, the tray portions 22 comprise transparent co-polyester that has been tinted with a light blue coloring. An example of a suitable co-polyester material is “Eastar 6763 PETG”, manufactured by the Eastman Chemical Company. Other suitable materials include ABS, Styrene, Vinyl, Olefin, Nylon and Urethane. The tray portions 22 are preferably formed by an injection molding process. However, it should be understood that the particular type of material and method of manufacture are not critical to the present invention. For example, the tray portions 22 may be formed of an opaque material, such as opaque plastic. An opaque material may have the advantage of protecting the medical device 100 from exposure to UV light, which may adversely affect the medical device 100 (e.g. by activating or drying out the adhesive coating 104). In addition, the tray portions 22 may further comprise release agents and other coatings such as silicone. Other types of medical devices 100 may require the use other materials or manufacturing methods. Other suitable materials and methods of manufacturing the packaging tray 10 are well known to those skilled in the art.

The tray portions 22 may comprise other features that improve the strength or handling of the packaging tray 10. For example, the tray portions 22 may include depressions or raised portions, such as those shown adjacent to the protective chamber 18, that are configured to increase the strength thereof and/or prevent planar surfaces of the tray portions 22 from excessive bending or flexing. Similarly, the tray portions 22 may comprises roughened surfaces to improve the tactile handling of the packaging tray 10. Such features are well known to those skilled in the art.

As discussed above, the illustrated embodiment of the packaging tray 10 of the present invention is manufactured by injection molding two identical tray portions 22. As shown in FIG. 8, one of the tray portions 22 (e.g., the top portion 14) is inverted and positioned above the other tray portion 22 (e.g., the bottom portion 16). The medical device 100 is then position between the two tray portions 22 so that the coated portion 104 and uncoated portion 106 is aligned with the protective chamber 18 and the anchoring region 20, respectively, as defined therebetween. The two tray portions 22 are then brought together until the attachment portions 24 are engaged and the medical device 100 is captured therebetween. Alternatively, the two tray portions 22 are first assembled together, and then the medical device 100 is inserted into the assembled packing tray 10. The assembled packaging tray 10, with the medical device 100 disposed therein, may then be further enclosed within a second or outer packaging material. In particular, the assembled packaging tray 10 is preferably disposed in a sealed outer pouch, outer box and/or other outer protective container (not shown), which may be configured to protect the packaging tray 10 and medical device 100 from contamination or mishandling.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area in view of the present disclosure. Illustrative embodiments of the present invention have been described in sufficient detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention.

Unless otherwise indicated, all ordinary words and terms used herein shall take their customary meaning as defined in The New Shorter Oxford English Dictionary, 1993 edition. All technical terms shall take on their customary meaning as established by the appropriate technical discipline utilized by those normally skilled in that particular art area. All medical terms shall take their meaning as defined by Stedman's Medical Dictionary, 27th edition.

1. A package for holding a medical device, comprising:
a medical device comprising a substantially flat configuration and having a portion of at least one surface thereof comprising a coating of an agent disposed thereupon, the medical device further comprising an uncoated portion thereof extending laterally from the portion of the at least one surface comprising the agent; and
a container unit having a top portion and a bottom portion that define a protective chamber therebetween configured to receive the coated portion of the medical device, the container unit further configured to include an anchoring region disposed lateral to the protective chamber and between the top and bottom portions such that when the medical device is disposed within the container unit, the uncoated portion is at least partially received securely within the anchoring region, causing the coated portion to be maintained within the protective chamber such that the at least one surface comprising the coating of the agent is prevented from contacting the top and bottom portions of the container unit adjacent thereto.

2. The package of claim 1 wherein the container unit is configured to facilitate egress of the medical device therefrom by an act of grasping the uncoated portion and sliding the medical device laterally from the container unit such that the at least one surface comprising the agent does not come into contact with the container unit during egress.

3. The package of claim 1 wherein the medical device comprises a first surface and a second surface disposed oppositely thereof, each of the first and second surfaces comprising the coating of the agent, and wherein the container unit is configured to maintain the medical device substantially centered within the protective chamber such that both the first and second surfaces are prevented from contacting the top and bottom portions of the container unit.

4. The package of claim 1 wherein the agent is one of an adhesive, a medicament, a pharmacologically active material and a biological material.

5. The package of claim 1 wherein the container unit comprises a tray configured such that the top portion joins together with the bottom portion such that a pair of slots comprising the anchoring region is created therebetween, each slot extending laterally from the protective chamber and being configured to receive and secure one of a pair of laterally extending tabs disposed on the medical device.

6. The package of claim 5 wherein the pair of laterally extending tabs each comprise a secured portion and a grasping portion, the secured portion being disposed within the anchoring region, and the grasping portion being exposed to an exterior of the container unit.

7. The package of claim 1 wherein the medical device comprises an implantable material removably disposed on an applicator strip, the applicator strip comprises the uncoated portion, and the coating of the agent is disposed on the implantable material.

8. The package of claim 7 wherein the applicator strip comprises a central portion and a pair of laterally extending tabs, the implantable material being removably disposed about the central portion, further wherein the central portion and the implantable material is disposed within the protective chamber and the pair of laterally extending tabs is disposed within the anchoring region.

9. The package of claim 1 wherein the top and bottom portions of the container unit comprise separate and identical tray portions.

10. The package of claim 9 wherein the top and bottom portions of the container unit are connected together by a plurality of attachment portions.

11. The package of claim 10 wherein the plurality of attachment portions comprise at least one of an adhesive and snap-fit connectors.

12. The package of claim 1 wherein the top and bottom portions of the container unit comprise a unitary construction.

13. The package of claim 1 wherein the uncoated portion of the medical device is partially exposed to an exterior of the container unit.

14. The package of claim 13 wherein the partially exposed portion is adjacent to the anchoring region.

15. The package of claim 1 wherein the uncoated portion of the medical device is secured within the anchoring region by friction.

16. The package of claim 1 wherein the container portion comprises a translucent material.

17. The package of claim 16 wherein the translucent material comprises a transparent plastic material.

18. A package for holding a medical device, wherein the medical device comprises a substantially flat configuration having a coated portion and an uncoated portion adjacent thereto, the package comprising:
a container unit comprising an interior surface defining a protective chamber, the protective chamber configured to receive the coated portion of the medical device therein, the container unit further comprising an anchoring region disposed adjacent to the protective chamber and configured to engage the uncoated portion of the medical device,
wherein the container unit is configured to support the medical device in a manner that prevents the coated portion from contacting the interior surface of the protective chamber.

19. The package of claim 18 further comprising a medical device, the medical device comprising a substantially flat configuration having a coated portion and an uncoated portion adjacent thereto, wherein the coated portion is disposed within the protective chamber of the container unit and the uncoated portion is at least partially secured within the anchoring region.

20. The package of claim 19 wherein the medical device comprises an implantable material removably disposed on an applicator strip, the applicator strip comprises the uncoated portion, and the coating of the agent is disposed on the implantable material.

21. The package of claim 19 wherein the agent is one of an adhesive, a medicament, a pharmacologically active material and a biological material.

22. The package of claim 18 wherein the container unit comprises a top portion connected to a bottom portion so as to define the protective chamber and the anchoring region therebetween.

23. The package of claim 22 wherein the top and bottom portions of the container unit comprise separate and identical tray portions that are connected together by a plurality of attachment portions, the plurality of attachment portions comprising at least one of an adhesive and snap-fit connectors.

24. A package for holding a medical device, comprising:
a medical device comprising an applicator strip, an implantable material removably attached about a central portion of the applicator strip, and a coating of an agent disposed on the implantable material, the applicator strip comprising a substantially flat configuration and having a grasping portion extending laterally from the central portion and adjacent to the implantable material; and
a container unit having an top portion and a bottom portion that define a protective chamber and an anchoring region therebetween, the anchoring region being disposed adjacent to the protective chamber, the protective chamber configured to receive the central portion of the applicator strip and the implantable material, the anchoring region configured to receive and frictionally engage the grasping portion of the applicator strip,
wherein the implantable material is prevented from contacting the top and bottom portions of the container unit adjacent to the protective chamber.
